(19) United States
(12) Patent Application Publication
(10) Pub. No.: US 2025/0204764 A1
(43) Pub. Date: Jun. 26, 2025

(54) IMAGING SYSTEM, ENDOSCOPE, ENDOSCOPE SYSTEM, AND IMAGING METHOD

(71) Applicant: OLYMPUS MEDICAL SYSTEMS CORP., Tokyo (JP)

(72) Inventor: Daiki YAMAMURA, Machida-shi (JP)

(73) Assignee: OLYMPUS MEDICAL SYSTEMS CORP., Tokyo (JP)

(21) Appl. No.: 18/975,123

(22) Filed: Dec. 10, 2024

Related U.S. Application Data
(60) Provisional application No. 63/612,803, filed on Dec. 20, 2023.

(51) Int. Cl. A61B 1/05; A61B 1/00

(52) U.S. Cl. CPC A61B 1/05; A61B 1/00002

(57) ABSTRACT

An imaging system includes an image sensor and a reception device. The image sensor outputs a reset signal and a pixel signal to a signal line. The reception device includes a reception circuit, an abnormality determination circuit, a correction circuit, and a signal generation circuit. The reception circuit receives the reset signal and the pixel signal. The abnormality determination circuit determines whether a value of the reset signal is abnormal. The correction circuit corrects the value of the reset signal when the abnormality determination circuit determines that the value of the reset signal is abnormal. The signal generation circuit generates a difference signal indicating a difference between the reset signal having the value corrected by the correction circuit and the pixel signal.

Priority is claimed on U.S. Provisional Patent Application No. 63/612,803, filed on Dec. 20, 2023, the content of which is incorporated herein by reference.

### BACKGROUND OF THE INVENTION

### Field of the Invention

The present invention relates to an imaging system, an endoscope, an endoscope system, and an imaging method.

### Description of Related Art

An endoscope having a complementary metal-oxide-semiconductor (CMOS) image sensor (hereinafter referred to as an image sensor) has been developed. The image sensor includes two or more pixels. Each pixel outputs a reset signal when a floating diffusion of the pixel is reset. Moreover, each pixel outputs a pixel signal in accordance with electric charge accumulated in the floating diffusion.

Reset noise is generated through a reset operation in each pixel and is superimposed on a reset signal and a pixel signal. Correlated double sampling (CDS) processing is executed to generate a difference between the reset signal and the pixel signal, and a video signal with reduced reset noise is generated. Two methods have been considered as methods of executing the CDS processing.

In a first method, a circuit in the image sensor performs the CDS processing and generates a difference signal indicating the difference between the reset signal and the pixel signal. The image sensor alternately outputs a difference signal and a reference voltage. A circuit other than the image sensor performs the CDS processing and generates a video signal that is a difference between the difference signal and the reference voltage.

In a second method, the image sensor alternately outputs a combination of a reset signal and a reference voltage and a combination of a pixel signal and a reference voltage. A circuit other than the image sensor executes the CDS processing for the reset signal and the reference voltage and generates a first difference signal indicating a difference between the reset signal and the reference voltage. The circuit other than the image sensor performs the CDS processing for the pixel signal and the reference voltage and generates a second difference signal indicating a difference between the pixel signal and the reference voltage. The circuit other than the image sensor performs the CDS processing for the first difference signal and the second difference signal and generates a video signal indicating a difference between the first difference signal and the second difference signal.

In the second method, the image sensor does not need to have a circuit that executes the CDS processing. Therefore, in the second method, the image sensor is miniaturized compared to the first method.

A device that generates a high-frequency signal, for example, an electrical scalpel may be used simultaneously with an endoscope. When this device is used, high-frequency noise is superimposed on a signal transmitted in a cable of the endoscope. The device disclosed in Japanese Unexamined Patent Application, First Publication No. 2010-142464 detects an operation of an electrical scalpel and removes noise superimposed on an image signal.

### SUMMARY OF THE INVENTION

According to a first aspect of the present invention, an imaging system has an image sensor and a reception device connected to the image sensor through a signal line. The image sensor outputs a reset signal and a pixel signal to the signal line. The reception device includes a reception circuit, an abnormality determination circuit, a correction circuit, and a signal generation circuit. The reception circuit receives the reset signal and the pixel signal output to the signal line. The abnormality determination circuit determines whether a value of the reset signal is abnormal using a first reference value. The correction circuit corrects the value of the reset signal when the abnormality determination circuit determines that the value of the reset signal is abnormal. The signal generation circuit generates a difference signal indicating a difference between the reset signal having the value corrected by the correction circuit and the pixel signal.

According to a second aspect of the present invention, in the first aspect, the reception device may include an analog-to-digital conversion circuit configured to convert the reset signal that is an analog signal into a digital reset signal that is a digital signal and convert the pixel signal that is an analog signal into a digital pixel signal that is a digital signal. The abnormality determination circuit may determine whether a value of the digital reset signal is abnormal using the value of the digital reset signal and the first reference value. The correction circuit may correct the value of the digital reset signal when the abnormality determination circuit determines that the value of the digital reset signal is abnormal. The signal generation circuit may generate a digital difference signal indicating a difference between the digital reset signal having the value corrected by the correction circuit and the digital pixel signal. The digital difference signal may be the difference signal.

According to a third aspect of the present invention, in the first aspect, the first reference value may include a maximum value and a minimum value of signals previously output as the reset signal. When the value of the reset signal is outside a range from the minimum value to the maximum value, the abnormality determination circuit may determine that the value of the reset signal is abnormal.

According to a fourth aspect of the present invention, in the first aspect, the image sensor may include two or more pixels including a target pixel and a peripheral pixel other than the target pixel. The abnormality determination circuit may determine whether a value of the reset signal output from the target pixel is abnormal using the value of the reset signal output from the target pixel and the first reference value. The first reference value may be a value of the reset signal output from the peripheral pixel.

According to a fifth aspect of the present invention, in the first aspect, the image sensor may output the reset signal and the pixel signal in each of two or more frame periods including a target frame period. The abnormality determination circuit may determine whether a value of the reset signal output in the target frame period is abnormal using the value of the reset signal output in the target frame period and the first reference value. The first reference value may be a value of the reset signal output in a frame period preceding the target frame period.

According to a sixth aspect of the present invention, in the first aspect, the abnormality determination circuit may determine whether a value of the pixel signal is abnormal using the value of the pixel signal and a second reference value. The correction circuit may correct the value of the pixel signal when the abnormality determination circuit determines that the value of the pixel signal is abnormal. The signal generation circuit may generate the difference signal indicating the difference between the reset signal having the value corrected by the correction circuit and the pixel signal having the value corrected by the correction circuit.

According to a seventh aspect of the present invention, in the sixth aspect, the image sensor may include two or more pixels including a target pixel and a peripheral pixel other than the target pixel. The abnormality determination circuit may determine whether a value of the pixel signal output from the target pixel is abnormal using the value of the pixel signal output from the target pixel and the second reference value. The second reference value may be the value of the pixel signal output from the peripheral pixel.

According to an eighth aspect of the present invention, in the sixth aspect, the image sensor may output the reset signal and the pixel signal in two or more frame periods including a target frame period. The abnormality determination circuit May determine whether a value of the reset signal output in the target frame period is abnormal using the value of the pixel signal output in the target frame period and the second reference value. The second reference value may be a value of the pixel signal output in a frame period preceding the target frame period.

According to a ninth aspect of the present invention, an endoscope includes a scope configured to be inserted into a living body. The scope includes an image sensor arranged on a distal end of the scope; and a connector connected to the image sensor through a signal line. The image sensor includes two or more pixels and an output circuit. Each of the two or more pixels outputs a reset signal and a pixel signal. The output circuit outputs the reset signal and the pixel signal to the signal line. The connector includes a reception circuit, an abnormality determination circuit, a correction circuit, and a signal generation circuit. The reception circuit receives the reset signal and the pixel signal output to the signal line. The abnormality determination circuit determines whether a value of the reset signal is abnormal using the value of the reset signal and a reference value. The correction circuit corrects the value of the reset signal when the abnormality determination circuit determines that the value of the reset signal is abnormal. The signal generation circuit generates a difference signal indicating a difference between the reset signal having the value corrected by the correction circuit and the pixel signal. The connector is connected to a signal-processing device configured to process the difference signal and output the processed difference signal to a monitor.

According to a tenth aspect of the present invention, an endoscope system includes a scope configured to be inserted into a living body; and a signal-processing device. The scope includes an image sensor arranged on a distal end of the scope; and a connector connected to the image sensor through a signal line. The image sensor outputs a reset signal and a pixel signal to the signal line. The connector includes a reception circuit configured to receive the reset signal and the pixel signal output to the signal line. The signal-processing device includes an abnormality determination circuit, a correction circuit, a signal generation circuit, and a signal-processing circuit. The abnormality determination circuit determines whether a value of the reset signal is abnormal using a reference value. The correction circuit corrects the value of the reset signal when the abnormality determination circuit determines that the value of the reset signal is abnormal. The signal generation circuit generates a difference signal indicating a difference between the reset signal having the value corrected by the correction circuit and the pixel signal. The signal-processing circuit processes the difference signal and outputs the processed difference signal to a monitor.

According to an eleventh aspect of the present invention, an imaging method includes the following: sequentially outputting a reset signal and a pixel signal output from each of two or more pixels included in an image sensor to a signal line; receiving the reset signal and the pixel signal output to the signal line; determining whether a value of the reset signal is abnormal using a reference value; correcting the value of the reset signal when it is determined that the value of the reset signal is abnormal; and generating a difference signal indicating a difference between the reset signal having the corrected value and the pixel signal.

### BRIEF DESCRIPTION OF THE DRAWINGS

FIG. 1 is a schematic diagram showing a configuration of an endoscope system according to a first embodiment of the present invention.

FIG. 2 is a block diagram showing a configuration of a camera unit, a connector unit, and a control unit included in the endoscope system according to the first embodiment of the present invention.

FIG. 3 is a block diagram showing a configuration of an image sensor included in the endoscope system according to the first embodiment of the present invention.

FIG. 4 is a diagram showing a configuration of a pixel in the image sensor included in the endoscope system according to the first embodiment of the present invention.

FIG. 5 is a flowchart showing an imaging method performed by the endoscope system according to the first embodiment of the present invention.

FIG. 6 is a timing chart showing waveforms of signals output to a video signal line in the first embodiment of the present invention.

FIG. 7 is a flowchart showing a procedure of abnormality detection processing in the first embodiment of the present invention.

FIG. 8 is a block diagram showing a configuration of a camera unit, a connector unit, and a control unit included in an endoscope system according to a second embodiment of the present invention.

### DETAILED DESCRIPTION OF THE INVENTION

Embodiments of the present invention will be described with reference to the drawings. Hereinafter, an example in which an endoscope system is used as an imaging system will be described.

### First Embodiment

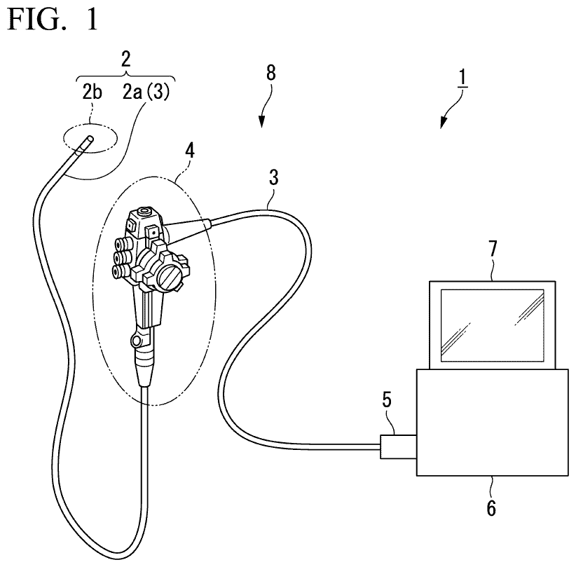

FIG. 1 shows a configuration of an endoscope system 1 of a first embodiment of the present invention. The endoscope system 1 shown in FIG. 1 includes an endoscope insertion unit 2, a transmission cable 3, an operation unit 4, a connector unit 5, a control unit 6, and a display device 7. The endoscope insertion unit 2, the transmission cable 3, the operation unit 4, and the connector unit 5 constitute a scope 8 (an endoscope).

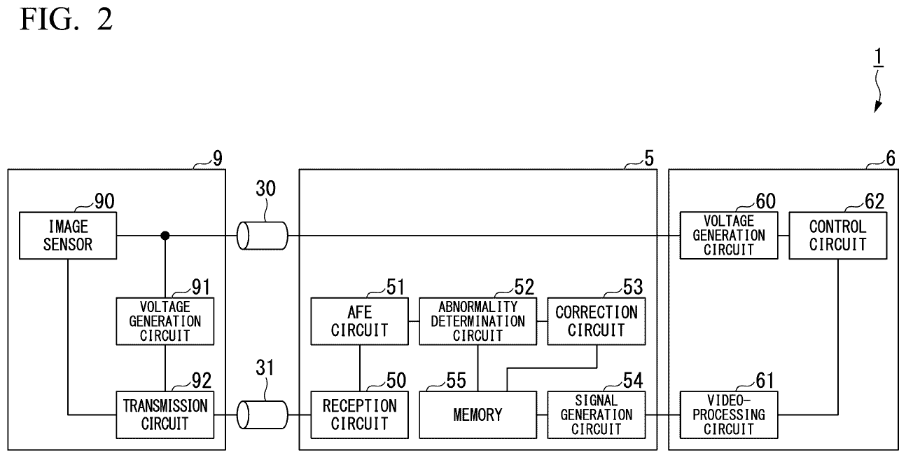

The endoscope insertion unit 2 has an insertion unit 2a. The insertion unit 2a is a part of the transmission cable 3. The insertion unit 2a is inserted into a living body, which is a subject. The endoscope insertion unit 2 generates a reset signal and a pixel signal by imaging the inside of the subject. The endoscope insertion unit 2 outputs the generated reset signal and the generated pixel signal to the connector unit 5. A camera unit 9 shown in FIG. 2 is arranged at a distal end 2b of the insertion unit 2a. In the insertion unit 2a, the operation unit 4 is connected to an end opposite to the distal end 2b. The operation unit 4 receives various kinds of operations on the endoscope insertion unit 2 from the user.

The transmission cable 3 connects the camera unit 9 and the connector unit 5.

The reset signal and the pixel signal generated by the camera unit 9 are output to the connector unit 5 via the transmission cable 3.

The connector unit 5 is connected to the transmission cable 3 and the control unit 6. The connector unit 5 processes the reset signal and the pixel signal output from the endoscope insertion unit 2 and generates a video signal. The connector unit 5 outputs the video signal to the control unit 6.

The control unit 6 performs image processing on the video signal output from the connector unit 5. Furthermore, the control unit 6 generally controls the entire endoscope system 1.

The display device 7 is, for example, a liquid crystal monitor, and displays a video based on a video signal processed by the control unit 6. Moreover, the display device 7 displays various kinds of information on the endoscope system 1.

The endoscope system 1 includes a camera unit 9, a connector unit 5, and a control unit 6 shown in FIG. 2. FIG. 2 shows a configuration of the camera unit 9, the connector unit 5, and the control unit 6. The camera unit 9 is disposed at the distal end 2b of the scope 8. The connector unit 5 constitutes a reception device. The operation unit 4 and the display device 7 are not shown in FIG. 2. The transmission cable 3 shown in FIG. 1 includes a power source line 30 and a video signal line 31 shown in FIG. 2.

The endoscope system 1 has a light source device that generates illumination light emitted to the subject. The light source device is not shown in FIG. 2.

The camera unit 9 includes an image sensor 90, a voltage generation circuit 91, and a transmission circuit 92 (an output circuit). The connector unit 5 includes a reception circuit 50, an analog front-end (AFE) circuit 51, an abnormality determination circuit 52, a correction circuit 53, a signal generation circuit 54, and a memory 55. The control unit 6 includes a voltage generation circuit 60, a video-processing circuit 61, and a control circuit 62.

A schematic configuration of the endoscope system 1 will be described. The connector unit 5 is connected to the image sensor 90 through the video signal line 31.

The image sensor 90 has two or more pixels, each of which outputs a reset signal and a pixel signal. The transmission circuit 92 outputs the reset signal and the pixel signal to the video signal line 31. The reception circuit 50 receives the reset signal and the pixel signal output to the video signal line 31. The abnormality determination circuit 52 determines whether a value of the reset signal is abnormal using the value of the reset signal and a first reference value. When the abnormality determination circuit 52 determines that the value of the reset signal is abnormal, the correction circuit 53 corrects the value of the reset signal. The signal generation circuit 54 generates a video signal (a difference signal) indicating a difference between the reset signal having the value corrected by the correction circuit 53 and the pixel signal.

Moreover, the abnormality determination circuit 52 determines whether the value of the pixel signal is abnormal using the value of the pixel signal and the second reference value. When the abnormality determination circuit 52 determines that the value of the pixel signal is abnormal, the correction circuit 53 corrects the value of the pixel signal. The signal generation circuit 54 generates a video signal indicating a difference between the reset signal having the value corrected by the correction circuit 53 and the pixel signal having the value corrected by the correction circuit 53.

A detailed configuration of the endoscope system 1 will be described. For example, the voltage generation circuit 60 is a voltage regulator. The voltage generation circuit 60 generates a power source voltage that is a DC voltage and outputs the power source voltage to the power source line 30. The power source line 30 is a signal line arranged in the transmission cable 3. The power source line 30 transfers the power source voltage to the camera unit 9.

The power source voltage transferred through the power source line 30 is input to the image sensor 90 and the voltage generation circuit 91 of the camera unit 9. The image sensor 90 generates a reset signal and a pixel signal based on the power source voltage and outputs the reset signal and the pixel signal to the transmission circuit 92. The voltage generation circuit 91 generates a reference voltage based on the power source voltage and outputs the reference voltage to the transmission circuit 92. The reference voltage is higher than a ground voltage and lower than the power source voltage. The reference voltage may be the same as the power source voltage.

The transmission circuit 92 transmits the reset signal and the reference voltage to the connector unit 5 by alternately outputting the reset signal and the reference voltage to the video signal line 31. Moreover, the transmission circuit 92 transmits the pixel signal and the reference voltage to the connector unit 5 by alternately outputting the pixel signal and the reference voltage to the video signal line 31.

The video signal line 31 is a signal line arranged in the transmission cable 3. The video signal line 31 transfers the reset signal and the reference voltage to the control unit 6 and transfers the pixel signal and the reference voltage to the control unit 6.

When a device that generates a high-frequency signal, for example, an electrical scalpel is used, noise including a fundamental frequency component and a high-frequency component (for example, several hundreds of megahertz (MHz) or more) is transmitted to the transmission cable 3. The noise is superimposed on a signal passing through the video signal line 31.

The reception circuit 50 receives the reset signal and the reference voltage transferred by the video signal line 31 and outputs the reset signal and the reference voltage to the AFE circuit 51. Moreover, the reception circuit 50 receives the pixel signal and the reference voltage transferred by the video signal line 31 and outputs the pixel signal and the reference voltage to the AFE circuit 51.

The AFE circuit 51 performs the CDS processing for the reset signal and the reference voltage and generates a signal indicating the difference between the reset signal and the reference voltage. Moreover, the AFE circuit 51 executes the CDS processing for the pixel signal and the reference voltage and generates a signal indicating the difference between the pixel signal and the reference voltage. Hereinafter, a signal generated through the CDS processing for the reset signal and the reference voltage is referred to as an analog reset signal. Moreover, hereinafter, a signal generated through the CDS processing for the pixel signal and the reference voltage is referred to as an analog pixel signal.

The AFE circuit 51 functions as an analog-to-digital (AD) conversion circuit configured to convert an analog signal into a digital signal. The AFE circuit 51 converts an analog reset signal generated through the CDS processing into a digital reset signal. Moreover, the AFE circuit 51 converts an analog pixel signal generated through the CDS processing into a digital pixel signal. The AFE circuit 51 outputs the digital reset signal and the digital pixel signal to the abnormality determination circuit 52.

The abnormality determination circuit 52 determines whether each of the digital reset signal and the digital pixel signal is abnormal using the values of the digital reset signal and the digital pixel signal and the reference value. When the abnormality determination circuit 52 determines that the value of the digital reset signal is abnormal, the correction circuit 53 corrects the value of the digital reset signal. When the abnormality determination circuit 52 determines that the value of the digital pixel signal is abnormal, the correction circuit 53 corrects the value of the digital pixel signal. The correction circuit 53 stores the digital reset signal and the digital pixel signal in the memory 55. Details of the process executed by the abnormality determination circuit 52 and the correction circuit 53 will be described below.

The memory 55 is a frame memory that stores the digital reset signal and the digital pixel signal. Moreover, the memory 55 stores the reference value that is used by the abnormality determination circuit 52.

The signal generation circuit 54 reads the digital reset signal and the digital pixel signal from the memory 55 and generates a video signal indicating the difference between the digital reset signal and the digital pixel signal. The signal generation circuit 54 outputs the video signal to the video-processing circuit 61.

The video-processing circuit 61 performs demosaicing processing, white balance adjustment processing, gamma correction processing, and the like for the video signal. The video-processing circuit 61 outputs the video signal to the display device 7.

At least one of the abnormality determination circuit 52, the correction circuit 53, the signal generation circuit 54, and the video-processing circuit 61 may be configured as a digital circuit including at least one of a processor and a logic circuit. For example, the processor is a central processing unit (CPU). For example, the logic circuit is at least one of an application-specific integrated circuit (ASIC) and a field-programmable gate array (FPGA). At least one of the abnormality determination circuit 52, the correction circuit 53, the signal generation circuit 54, and the video-processing circuit 61 may include one or more processors. At least one of the abnormality determination circuit 52, the correction circuit 53, the signal generation circuit 54, and the video-processing circuit 61 may include one or more logic circuits.

A computer of the connector unit 5 or the control unit 6 may read the program and execute the read program. The program includes commands for defining the operation of at least one of the abnormality determination circuit 52, the correction circuit 53, the signal generation circuit 54, and the video-processing circuit 61. That is, the function of at least one of the abnormality determination circuit 52, the correction circuit 53, the signal generation circuit 54, and the video-processing circuit 61 may be realized by software. The program may be transmitted from a computer holding the program to the connector unit 5 or the control unit 6 via a transmission medium or by a transmission wave in the transmission medium. The “transmission medium” for transmitting the program is a medium having a function of transmitting information. Media having a function of transmitting information include a network (a communication network) such as the Internet or a communication circuit (a communication line) such as a telephone circuit. The above-described program may realize some of the above-described functions. Furthermore, the above-described program may be a so-called differential file (differential program). The above-described function may be realized by a combination of the differential program and a program already recorded on the computer.

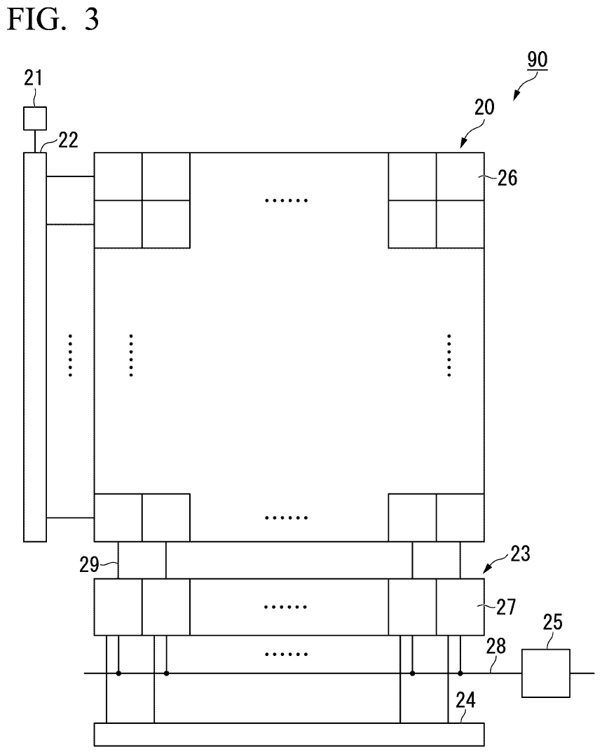

FIG. 3 shows a configuration of the image sensor 90. The image sensor 90 includes an imaging unit 20, a timing generator 21, a vertical selection circuit 22, a column circuit unit 23, a horizontal selection circuit 24, and an output unit 25.

The imaging unit 20 has two or more pixels 26 arranged in a matrix shape. The two or more pixels 26 form an array of m rows and n columns. The number (m) of rows is 2 or more and the number (n) of columns is 2 or more. The number of rows and the number of columns are not necessarily the same. Each pixel 26 outputs a reset signal having a reset level and a pixel signal having a signal level.

The timing generator 21 generates a timing signal and outputs the timing signal to the vertical selection circuit 22. The vertical selection circuit 22 selects pixels 26 arranged in a row direction in an array of the two or more pixels 26. The vertical selection circuit 22 controls an operation of the selected pixel 26. The vertical selection circuit 22 outputs a control signal for controlling the two or more pixels 26 for each row in the array of the two or more pixels 26.

The column circuit unit 23 has two or more column circuits 27. Each column circuit 27 is arranged for each column in the array of the two or more pixels 26. Each column circuit 27 is connected to a vertical signal line 29 extending in a vertical direction, i.e., a column direction. The vertical signal line 29 is arranged for each column in the array of the two or more pixels 26. The vertical signal line 29 is connected to pixels 26 of each column. Each column circuit 27 is electrically connected to each pixel 26 via the vertical signal line 29. Each column circuit 27 holds a reset signal and a pixel signal output from each pixel 26.

Each column circuit 27 is connected to a horizontal signal line 28 extending in the horizontal direction, i.e., the row direction. A selection pulse is output from the horizontal selection circuit 24 to each column circuit 27. The column circuit 27 selected based on the selection pulse outputs a reset signal and a pixel signal to the horizontal signal line 28.

One column circuit 27 may be arranged for each of the two or more columns in the array of the two or more pixels 26, and the one column circuit 27 may be used in time division in the two or more columns. Therefore, the column circuit 27 has only to be arranged so as to correspond to one or more columns in the array of the two or more pixels 26.

The horizontal signal line 28 is connected to the output unit 25. The horizontal selection circuit 24 sequentially selects the column circuits 27 by sequentially outputting selection pulses to the column circuits 27. The reset signal and the pixel signal output from the column circuit 27 selected by the horizontal selection circuit 24 are transferred to the output unit 25. The output unit 25 outputs the reset signal and the pixel signal to the transmission circuit 92.

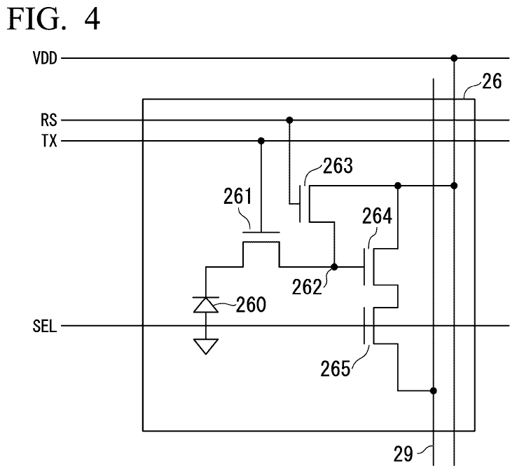

FIG. 4 shows a configuration of each pixel 26. The pixel 26 shown in FIG. 4 includes a photoelectric conversion element 260, a transfer transistor 261, a floating diffusion (FD) 262, a reset transistor 263, an amplification transistor 264, and a selection transistor 265.

The photoelectric conversion element 260 is a photodiode. The photoelectric conversion element 260 photoelectrically converts light incident on the photoelectric conversion element 260 and generates electric charge corresponding to the amount of the light. The transfer transistor 261 transfers the electric charge generated by the photoelectric conversion element 260 to the FD 262. The FD 262 holds the electric charge transferred by the transfer transistor 261.

The reset transistor 263 resets a voltage of the FD 262 to a voltage corresponding to a power source voltage VDD. Thereby, the reset transistor 263 resets the electric charge held in the FD 262. When the electric charge held in the FD 262 is reset, the amplification transistor 264 generates a reset signal by amplifying a signal based on the voltage of the FD 262. The selection transistor 265 outputs the reset signal to the vertical signal line 29.

After the electric charge held in the FD 262 is reset, the electric charge generated by the photoelectric conversion element 260 is transferred to the FD 262 and the FD 262 holds the electric charge. The amplification transistor 264 generates a pixel signal by amplifying a signal based on the voltage of the FD 262. The selection transistor 265 outputs the pixel signal to the vertical signal line 29.

The vertical selection circuit 22 outputs a reset control signal RS, a transfer control signal TX, and a selection control signal SEL. The reset control signal RS is supplied to the reset transistor 263. The transfer control signal TX is supplied to the transfer transistor 261. The selection control signal SEL is supplied to the selection transistor 265.

A state of each of the transfer transistor 261, the reset transistor 263, and the selection transistor 265 is one of an ON state and an OFF state. The state of each transistor can be switched between the ON and OFF states.

The state of the reset transistor 263 is controlled in accordance with the reset control signal RS. The state of the transfer transistor 261 is controlled in accordance with the transfer control signal TX. The state of the selection transistor 265 is controlled in accordance with the selection control signal SEL.

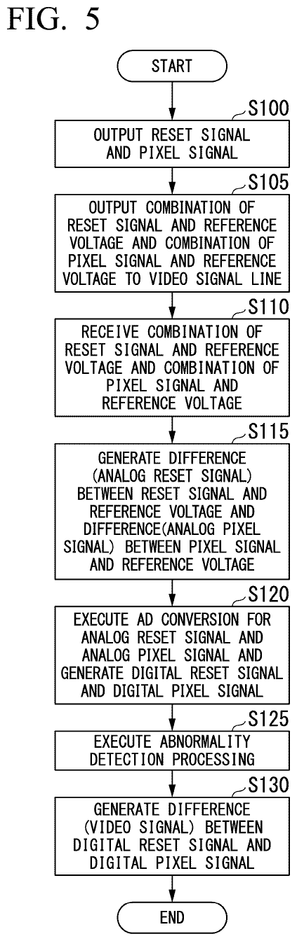

FIG. 5 shows an imaging method performed by the endoscope system 1. An operation of the endoscope system 1 will be described using FIG. 5.

The image sensor 90 performs imaging processing in each of two or more consecutive frame periods and outputs a reset signal and a pixel signal generated in each pixel 26 in each frame period. Hereinafter, processing using the reset signal and the pixel signal generated by each pixel 26 in one frame period will be described.

Each pixel 26 of the image sensor 90 outputs a reset signal to the vertical signal line 29 in a first period and outputs a pixel signal to the vertical signal line 29 in a second period subsequent to the first period (step S100).

Details of step S100 will be described. Hereinafter, the array of the two or more pixels 26 arranged in the imaging unit 20 is referred to as a pixel array. After the first period is started, the pixels 26 of each column in the first row of the pixel array output reset signals to the vertical signal line 29. The reset signals output from the pixels 26 of each column are output to the output unit 25 via the column circuit 27 and the horizontal signal line 28. The output unit 25 outputs the reset signals output from the pixels 26 of each row to the transmission circuit 92.

After the pixels 26 of all columns in the first row output reset signals to the vertical signal line 29, the pixels 26 of each column in the second row of the pixel array output reset signals to the vertical signal line 29. An operation similar to that described above is executed, and the output unit 25 outputs the reset signals output from the pixels 26 of each column in the second row to the transmission circuit 92. The above-described operation is iterated, and the output unit 25 outputs reset signals output from the pixels 26 of all rows and columns to the transmission circuit 92.

After the first period ends and the second period starts, the pixels 26 of each column in the first row of the pixel array outputs pixel signals to the vertical signal line 29. The pixel signals output from the pixels 26 of each column are output to the output unit 25 via the column circuit 27 and the horizontal signal line 28. The output unit 25 outputs the pixel signals output from the pixel 26 of each column to the transmission circuit 92.

After the pixels 26 of all columns in the first row output the pixel signals to the vertical signal line 29, the pixels 26 of each column in the second row of the pixel array output pixel signals to the vertical signal line 29. An operation similar to that described above is executed, and the output unit 25 outputs the pixel signals output from the pixel 26 of each column in the second row to the transmission circuit 92. The above-described operation is iterated, and the output unit 25 outputs pixel signals output from the pixels 26 of all rows and all columns to the transmission circuit 92.

A reset signal and a pixel signal of each pixel 26 are input from the output unit 25 to the transmission circuit 92. Moreover, a reference voltage is input from the voltage generation circuit 91 to the transmission circuit 92. The transmission circuit 92 alternately outputs the reset signal and the reference voltage to the video signal line 31. Moreover, the transmission circuit 92 alternately outputs the pixel signal and the reference voltage to the video signal line 31 (step S105).

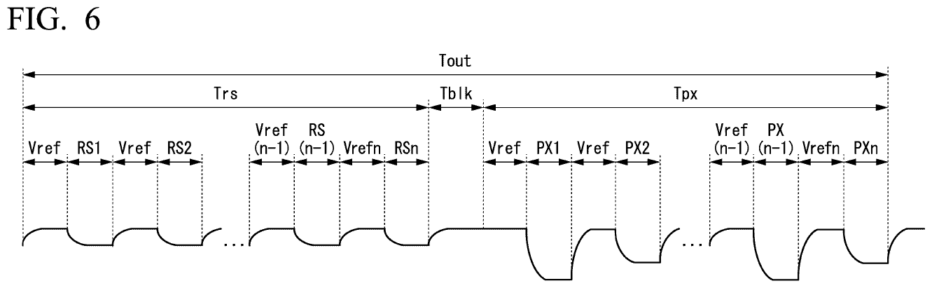

FIG. 6 shows waveforms of signals output from the transmission circuit 92 to the video signal line 31. Waveforms of the signals in an output period Tout is shown in FIG. 6. A reset signal and a pixel signal are output from each of the pixels 26 of one row of the pixel array in the output period Tout. One frame period includes the same number of output periods Tout as that of rows in the pixel array.

The operation of the transmission circuit 92 in the output period Tout allocated to the first row of the pixel array will be described. The output period Tout includes a reset output period Trs, a pixel output period Tpx, and a horizontal blanking period Tblk. The transmission circuit 92 outputs a reference voltage Vref and a reset signal RS1 to the video signal line 31 in the reset output period Trs. The reset signal RS1 is a reset signal output from the pixel 26 of the first row and the first column. For example, the length of a period in which the reference voltage Vref and the reset signal RS1 are output is 100 ns.

After the reference voltage Vref and the reset signal RS1 are output, the transmission circuit 92 outputs the reference voltage Vref and a reset signal RS2 to the video signal line 31 in the reset output period Trs. The reset signal RS2 is a reset signal output from the pixel 26 of the first row and the second row. The transmission circuit 92 iteratively outputs the reference voltage Vref and the reset signal to the video signal line 31 until the reset signals output from the pixels 26 in all columns of the first row are output to the video signal line 31.

After the reset output period Trs ends, the horizontal blanking period Tblk starts. The transmission circuit 92 stops the output of the reference voltage Vref and the reset signal in the horizontal blanking period Tblk.

After the horizontal blanking period Tblk ends, the transmission circuit 92 outputs the reference voltage Vref and the pixel signal PX1 to the video signal line 31 in the pixel output period Tpx. The pixel signal PX1 is a pixel signal output from the pixel 26 in the first row and the first column. For example, the length of the period in which the reference voltage Vref and the pixel signal PX1 are output is 100 ns.

After the reference voltage Vref and the pixel signal PX1 are output, the transmission circuit 92 outputs the reference voltage Vref and a pixel signal PX2 to the video signal line 31 in the pixel output period Tpx. The pixel signal PX2 is a pixel signal output from the pixel 26 in the first row and the second column. The transmission circuit 92 iteratively outputs the reference voltage Vref and the pixel signal to the video signal line 31 until the pixel signals output from the pixels 26 in all columns of the first row are output to the video signal line 31.

An operation of the endoscope system 1 will be described using FIG. 5 again. The reception circuit 50 receives the reset signal and the reference voltage output from the transmission circuit 92 and outputs the reset signal and the reference voltage to the AFE circuit 51. Moreover, the reception circuit 50 receives the pixel signal and the reference voltage output from the transmission circuit 92 and outputs the pixel signal and the reference voltage to the AFE circuit 51 (step S110).

The AFE circuit 51 executes the CDS processing for the reset signal and the reference voltage and generates an analog reset signal indicating a difference between the reset signal and the reference voltage. Moreover, the AFE circuit 51 executes the CDS processing for the pixel signal and the reference voltage and generates an analog pixel signal indicating a difference between the pixel signal and the reference voltage (step S115).

The AFE circuit 51 converts the analog reset signal into a digital reset signal by executing AD conversion processing for the analog reset signal. Moreover, the AFE circuit 51 converts the analog pixel signal into a digital pixel signal by executing AD conversion processing for the analog pixel signal. The AFE circuit 51 outputs the digital reset signal and the digital pixel signal to the abnormality determination circuit 52 (step S120).

The abnormality determination circuit 52 and the correction circuit 53 execute the abnormality detection processing using the digital reset signal and the digital pixel signal (step S125).

The digital reset signal and the digital pixel signal are stored in the memory 55 through the abnormality detection processing. The digital reset signal or the digital pixel signal may have a value corrected by the correction circuit 53.

The signal generation circuit 54 reads the digital reset signal and the digital pixel signal from the memory 55 and generates a video signal indicating the difference between the digital reset signal and the digital pixel signal. The signal generation circuit 54 outputs the video signal to the video-processing circuit 61 (step S130).

The video-processing circuit 61 performs predetermined video processing for the video signal and outputs the video signal to the display device 7. An operation of the video-processing circuit 61 is not shown in FIG. 5.

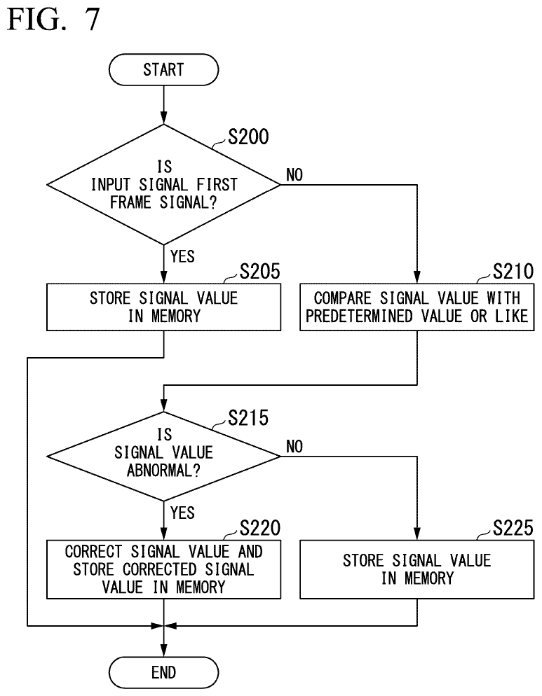

FIG. 7 shows a procedure of the abnormality detection processing in step S125. Operations of the abnormality determination circuit 52 and the correction circuit 53 in the abnormality detection processing will be described using FIG. 7.

Hereinafter, the abnormality detection processing for a digital reset signal will be described. FIG. 7 shows processing executed for a digital reset signal corresponding to a reset signal output from one pixel 26. The abnormality determination circuit 52 and the correction circuit 53 iterate the processing shown in FIG. 7 and execute the abnormality detection processing for digital reset signals corresponding to the reset signals output from all pixels 26.

The abnormality determination circuit 52 determines whether the digital reset signal input from the AFE circuit 51 is a signal of a first frame (step S200). The first frame corresponds to a reset signal output from the image sensor 90 in a first frame period.

When the abnormality determination circuit 52 determines that the digital reset signal is a signal of the first frame in step S200, the abnormality determination circuit 52 stores a value of the digital reset signal in the memory 55 (step S205). When step S205 is executed, the abnormality detection processing shown in FIG. 7 ends.

When the abnormality determination circuit 52 determines that the digital reset signal is not a signal of the first frame in step S200, the abnormality determination circuit 52 compares the value of the digital reset signal with a predetermined value or the like (step S210).

After step S210, the abnormality determination circuit 52 determines whether the value of the digital reset signal is abnormal based on a processing result of step S210 (step S215).

When the abnormality determination circuit 52 determines that the value of the digital reset signal is abnormal in step S215, the abnormality determination circuit 52 corrects the value of the digital reset signal and stores the corrected value in the memory 55 (step S220). When the abnormality determination circuit 52 determines that the value of the digital reset signal is not abnormal in step S215, the abnormality determination circuit 52 stores the value of the digital reset signal in the memory 55 (step S225). When step S220 or step S215 has been executed, the abnormality detection processing shown in FIG. 7 ends.

The abnormality determination circuit 52 and the correction circuit 53 execute abnormality detection processing for the digital pixel signal by executing processing similar to that described above. Although the abnormality detection processing for a digital reset signal and a digital pixel signal is executed in this example, the abnormality detection processing only for a digital reset signal may be executed.

Details of step S210, step S215, and step S220 will be described. Hereinafter, first to fifth examples will be described. A digital reset signal is used in the first to third examples, and a digital pixel signal is used in the fourth and fifth examples.

Hereinafter, one of the two or more frame periods is referred to as a target frame period. A digital reset signal corresponding to the reset signal output from the image sensor 90 in a specific frame period is referred to as a digital reset signal for the specific frame period. The value of the digital reset signal or the digital pixel signal corresponding to the reset signal or the pixel signal output from the specific pixel 26 is referred to as a signal value of the specific pixel 26. One pixel 26 in the pixel array is referred to as a target pixel.

A first example will be described. PVT noise generated in accordance with the manufacturing process, voltage, and temperature may be superimposed on the reset signal. The maximum and minimum values of the reset signal for the frame period preceding the target frame period are measured in advance, and the maximum and minimum values are stored in the memory 55. The maximum and minimum values correspond to the first reference value. The abnormality determination circuit 52 reads the maximum value and the minimum value from the memory 55 and compares the signal value of the target pixel with the maximum value and the minimum value in step S210.

When the signal value of the target pixel is greater than or equal to the minimum value and less than or equal to the maximum value, the abnormality determination circuit 52 determines that the signal value of the target pixel is not abnormal in step S215. In other words, when the signal value of the target pixel is in a range from the minimum value to the maximum value, the abnormality determination circuit 52 determines that the signal value of the target pixel is not abnormal in step S215. When the signal value of the target pixel is less than the minimum value or greater than the maximum value, the abnormality determination circuit 52 determines that the signal value of the target pixel is abnormal in step S215. In other words, when the signal value of the target pixel is outside the range from the minimum value to the maximum value, the abnormality determination circuit 52 determines that the signal value of the target pixel is abnormal in step S215.

When the signal value of the target pixel is less than the minimum value, the correction circuit 53 replaces the signal value of the target pixel with the minimum value in step S220. When the signal value of the target pixel is greater than the maximum value, the correction circuit 53 replaces the signal value of the target pixel with the maximum value in step S220.

In the first example, the abnormality determination circuit 52 and the correction circuit 53 use a digital reset signal in the target frame period. In the abnormality detection processing for the digital reset signal in the target frame period, the abnormality determination circuit 52 and the correction circuit 53 do not use a digital reset signal in the frame period preceding the target frame period. The memory 55 does not store any digital reset signals other than the digital reset signal in the target frame period. Therefore, the circuit scale of the abnormality determination circuit 52, the correction circuit 53, and the memory 55 is reduced.

A second example will be described. The abnormality determination circuit 52 calculates an average value of the signal values of two or more pixels near the target pixel in step S210. The two or more pixels are adjacent to the target pixel. For example, the abnormality determination circuit 52 calculates an average value between a signal value of a pixel on the left of the target pixel and a signal value of a pixel on the right of the target pixel. The average value corresponds to a first reference value. The abnormality determination circuit 52 may calculate an average value of a signal value of a pixel above the target pixel, a signal value of a pixel below the target pixel, a signal value of a pixel on the left of the target pixel, and a signal value of a pixel on the right of the target pixel. The abnormality determination circuit 52 may calculate an average value of signal values of eight pixels 26 adjacent to the target pixel.

In the second example, values of digital reset signals of some of the two or more pixels 26 in the pixel array are processed as the signal value of the target pixel described above. Values of digital reset signals corresponding to pixel signals output from the pixels 26 in the top row, the bottom row, the leftmost row, and the rightmost row are not processed as the signal value of the target pixel and are not stored in the memory 55.

The abnormality determination circuit 52 calculates a difference between the signal value of the target pixel and the average value in step S210. The abnormality determination circuit 52 compares an absolute value of the difference with a threshold value stored in the memory 55 in step S210.

When the absolute value is less than or equal to the threshold value, the abnormality determination circuit 52 determines that the signal value of the target pixel is not abnormal in step S215. When the absolute value is greater than the threshold value, the abnormality determination circuit 52 determines that the signal value of the target pixel is abnormal in step S215.

When the absolute value is greater than the threshold value, the correction circuit 53 replaces the signal value of the target pixel with the average value in step S220.

In the first example, when a digital reset signal on which noise is superimposed has a value between the minimum value and the maximum value, the noise is not removed. On the other hand, in the second example, when the absolute value of the difference calculated from the digital reset signal on which the noise is superimposed is greater than the threshold value, the noise is removed. Therefore, compared with the first example, an influence of noise is reduced.

In the second example, the memory 55 does not store any digital reset signals other than the digital reset signal in the target frame period. Therefore, the circuit scale of the memory 55 is reduced.

A third example will be described. The memory 55 stores digital reset signals in one or more frame periods preceding the target frame period. In step S210, the abnormality determination circuit 52 calculates a difference between a signal value of the target pixel in the target frame period and a signal value (a first reference value) of the same target pixel in a frame period immediately preceding the target frame period. Alternatively, in step S210, the abnormality determination circuit 52 calculates a difference between the signal value of the target pixel in the target frame period and an average value (a first reference value) between the signal values of the same target pixel in two or more frame periods preceding the target frame period.

The abnormality determination circuit 52 compares an absolute value of the difference with a threshold value stored in the memory 55 in step S210. When the absolute value is less than or equal to the threshold value, the abnormality determination circuit 52 determines that the value of the digital reset signal is not abnormal in step S215. When the absolute value is greater than the threshold value, the abnormality determination circuit 52 determines that the value of the digital reset signal is abnormal in step S215.

When the absolute value is greater than the threshold value, the correction circuit 53 replaces the signal value of the target pixel of the target frame period with the signal value of the target pixel in the frame period immediately preceding the target frame period in step S220. Alternatively, the correction circuit 53 replaces the signal value of the target pixel of the target frame period with the above-described average value in step S220.

In the third example, when the digital reset signal on which the noise is superimposed has a value between the minimum value and the maximum value, the noise is not removed. On the other hand, in the third example, when the absolute value of the difference calculated from the digital reset signal on which the noise is superimposed is greater than the threshold value, the noise is removed. Therefore, compared with the first example, an influence of noise is reduced.

In a fourth example, processing similar to that in the second example is executed for the digital pixel signal. Therefore, descriptions of the fourth example will be omitted.

In a fifth example, processing similar to that in the third example is performed for the digital pixel signal. Therefore, descriptions of the fifth example will be omitted.

An endoscope according to each aspect of the present invention includes the scope 8 that is inserted into a living body. The scope 8 includes the image sensor 90 arranged at the distal end 2b of the scope 8 and the connector unit 5 connected to the image sensor 90 through the video signal line 31. The image sensor 90 includes the two or more pixels 26 and the transmission circuit 92. Each of the two or more pixels 26 outputs a reset signal and a pixel signal. The transmission circuit 92 outputs the reset signal and the pixel signal to the video signal line 31. The reception circuit 50 receives the reset signal and the pixel signal output to the video signal line 31. The abnormality determination circuit 52 determines whether a signal value that is a value of the reset signal or the pixel signal is abnormal using the signal value and a reference value. When the abnormality determination circuit 52 determines that the signal value is abnormal, the correction circuit 53 corrects the signal value. The signal generation circuit 54 generates a video signal indicating a difference between the reset signal and the pixel signal. The connector unit 5 is connected to the control unit 6 (a signal processing device) configured to process the video signal and output the processed video signal to the display device 7.

An imaging method according to each aspect of the present invention includes first to fifth steps. In the first step (step S105), the transmission circuit 92 sequentially outputs a reset signal and a pixel signal output from each of the two or more pixels 26 to the video signal line 31. The reception circuit 50 receives the reset signal and the pixel signal in the second step (step S110). In the third step (steps S210 and S215), the abnormality determination circuit 52 determines whether a signal value that is a value of the reset signal or the pixel signal is abnormal using the signal value and a reference value. When it is determined that the signal value is abnormal, the correction circuit 53 corrects the signal value in the fourth step (step S220). In the fifth step (step S130), the signal generation circuit 54 generates a video signal indicating a difference between the reset signal and the pixel signal.

Each aspect of the present invention may include the following modified example. The connector unit 5 includes the AFE circuit 51 (an analog-to-digital conversion circuit) configured to convert a reset signal that is an analog signal into a digital reset signal that is a digital signal and convert a pixel signal that is an analog signal into a digital pixel signal that is a digital signal. The abnormality determination circuit 52 determines whether the value of the digital reset signal is abnormal using the value of the digital reset signal and a first reference value. When the abnormality determination circuit 52 determines that the value of the digital reset signal is abnormal, the correction circuit 53 corrects the value of the digital reset signal. The signal generation circuit 54 generates a video signal (a digital difference signal) indicating a difference between the digital reset signal having the value corrected by the correction circuit 53 and the digital pixel signal.

Each aspect of the present invention may include the following modified example. The abnormality determination circuit 52 determines whether the value of the digital pixel signal is abnormal using the value of the digital pixel signal and a second reference value. When the abnormality determination circuit 52 determines that the value of the digital pixel signal is abnormal, the correction circuit 53 corrects the value of the digital pixel signal. The signal generation circuit 54 generates a video signal indicating a difference between a digital reset signal having the value corrected by the correction circuit 53 and a digital pixel signal having the value corrected by the correction circuit 53.

Each aspect of the present invention may include the following modified example. The first reference value includes the maximum and minimum values of the signal previously output as a reset signal from each of the two or more pixels 26. When the value of the reset signal is outside a range from the minimum value to the maximum value, the abnormality determination circuit 52 determines that the value of the reset signal is abnormal.

Each aspect of the present invention may include the following modified example. The two or more pixels 26 include a target pixel and a peripheral pixel other than the target pixel. The abnormality determination circuit 52 determines whether a value of a reset signal output from the target pixel is abnormal using the value of the reset signal output from the target pixel and the first reference value. The first reference value is a value of a reset signal output from the peripheral pixel.

Each aspect of the present invention may include the following modified example. Each of the two or more pixels 26 outputs a reset signal and a pixel signal in each of the two or more frame periods including the target frame period. The abnormality determination circuit 52 determines whether the value of the reset signal output in the target frame period is abnormal using the value of the reset signal output in the target frame period and the first reference value. The first reference value is the value of the reset signal output in the frame period preceding the target frame period.

Each aspect of the present invention may include the following modified example. The abnormality determination circuit 52 determines whether a value of a pixel signal is abnormal using the value of the pixel signal and the second reference value. When the abnormality determination circuit 52 determines that the value of the pixel signal is abnormal, the correction circuit 53 corrects the value of the pixel signal. The signal generation circuit 54 generates a video signal indicating a difference between a reset signal having the value corrected by the correction circuit 53 and a pixel signal having the value corrected by the correction circuit 53.

Each aspect of the present invention may include the following modified example. The two or more pixels 26 include a target pixel and a peripheral pixel other than the target pixel. The abnormality determination circuit 52 determines whether the value of the pixel signal output from the target pixel is abnormal using the value of the pixel signal output from the target pixel and the second reference value. The second reference value is a value of a pixel signal output from the peripheral pixel.

Each aspect of the present invention may include the following modified example. Each of the two or more pixels 26 outputs a reset signal and a pixel signal in each of the two or more frame periods including the target frame period. The abnormality determination circuit 52 determines whether the value of the pixel signal output in the target frame period is abnormal using the value of the pixel signal output in the target frame period and the second reference value. The second reference value is a value of a pixel signal output in the frame period preceding the target frame period.

In the first embodiment, the abnormality determination circuit 52 determines whether the value of the reset signal or the pixel signal is abnormal. When the abnormality determination circuit 52 determines that the value of the reset signal or the pixel signal is abnormal, the correction circuit 53 corrects the value. Therefore, the endoscope system 1 can reduce an influence of noise on a signal output from the image sensor 90.

### Second embodiment

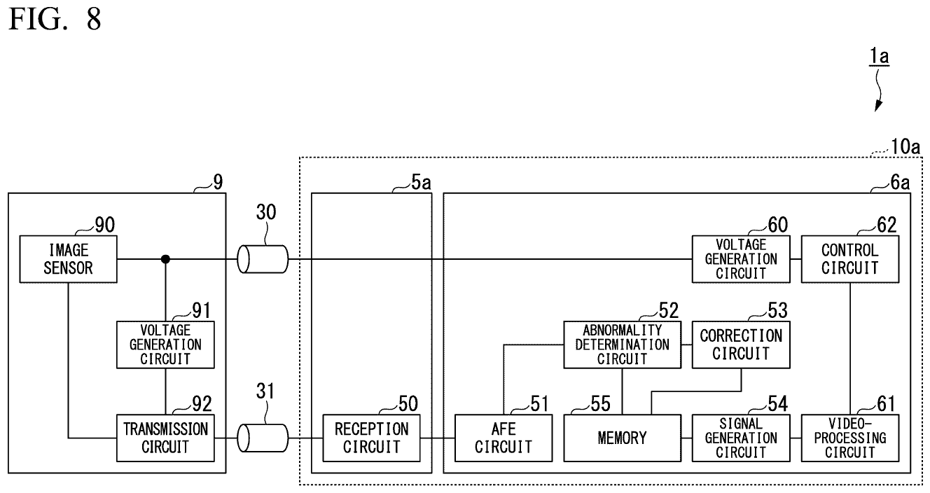

A second embodiment of the present invention will be described. The endoscope system 1 shown in FIG. 2 is changed to an endoscope system 1a shown in FIG. 8. FIG. 8 shows a configuration of the endoscope system 1a. Descriptions of the same configuration as that shown in FIG. 2 will be omitted.

The connector unit 5 shown in FIG. 2 is changed to a connector unit 5a shown in FIG. 8. The control unit 6 shown in FIG. 2 is changed to a control unit 6a shown in FIG. 8. The connector unit 5a and the control unit 6a constitute a reception device 10a.

As shown in FIG. 2, the AFE circuit 51, the abnormality determination circuit 52, the correction circuit 53, the signal generation circuit 54, and the memory 55 are arranged in the connector unit 5. As shown in FIG. 8, these are arranged in the control unit 6a.

An operation of the endoscope system la is similar to that of the endoscope system 1 in the first embodiment. Therefore, descriptions of the operation of the endoscope system la will be omitted.

The endoscope system la includes a scope that is inserted into a living body and the control unit 6a (a signal processing device). The scope includes an image sensor 90 arranged at a distal end 2b of the scope and the connector unit 5a connected to the image sensor 90 through a video signal line 31. The connector unit 5a has a reception circuit 50. The control unit 6a includes the abnormality determination circuit 52, the correction circuit 53, the signal generation circuit 54, and a video-processing circuit 61 (a signal processing circuit).

Like the endoscope system 1, the endoscope system la can reduce an influence of noise on a signal output from the image sensor 90.

While preferred embodiments of the present invention have been described and shown above, it should be understood that these are examples of the present invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An imaging system comprising:
an image sensor; and
a reception device connected to the image sensor through a signal line,
wherein the image sensor is configured to output a reset signal and a pixel signal to the signal line, and
wherein the reception device includes:
a reception circuit configured to receive the reset signal and the pixel signal output to the signal line;
an abnormality determination circuit configured to determine whether a value of the reset signal is abnormal using a first reference value;
a correction circuit configured to correct the value of the reset signal when the abnormality determination circuit determines that the value of the reset signal is abnormal; and
a signal generation circuit configured to generate a difference signal indicating a difference between the reset signal having the value corrected by the correction circuit and the pixel signal.

2. The imaging system according to claim 1,
wherein the reception device includes an analog-to-digital conversion circuit configured to convert the reset signal that is an analog signal into a digital reset signal that is a digital signal and convert the pixel signal that is an analog signal into a digital pixel signal that is a digital signal,
wherein the abnormality determination circuit is configured to determine whether a value of the digital reset signal is abnormal using the value of the digital reset signal and the first reference value,
wherein the correction circuit is configured to correct the value of the digital reset signal when the abnormality determination circuit determines that the value of the digital reset signal is abnormal,
wherein the signal generation circuit is configured to generate a digital difference signal indicating a difference between the digital reset signal having the value corrected by the correction circuit and the digital pixel signal, and
wherein the digital difference signal is the difference signal.

3. The imaging system according to claim 1,
wherein the first reference value includes a maximum value and a minimum value of signals previously output as the reset signal, and
wherein, when the value of the reset signal is outside a range from the minimum value to the maximum value, the abnormality determination circuit is configured to determine that the value of the reset signal is abnormal.

4. The imaging system according to claim 1,
wherein the image sensor includes two or more pixels including a target pixel and a peripheral pixel other than the target pixel,
wherein the abnormality determination circuit is configured to determine whether a value of the reset signal output from the target pixel is abnormal using the value of the reset signal output from the target pixel and the first reference value, and
wherein the first reference value is the value of the reset signal output from the peripheral pixel.

5. The imaging system according to claim 1,
wherein the image sensor is configured to output the reset signal and the pixel signal in each of two or more frame periods including a target frame period,
wherein the abnormality determination circuit is configured to determine whether a value of the reset signal output in the target frame period is abnormal using the value of the reset signal output in the target frame period and the first reference value, and
wherein the first reference value is a value of the reset signal output in a frame period preceding the target frame period.

6. The imaging system according to claim 1,
wherein the abnormality determination circuit is configured to determine whether a value of the pixel signal is abnormal using the value of the pixel signal and a second reference value,
wherein the correction circuit is configured to correct the value of the pixel signal when the abnormality determination circuit determines that the value of the pixel signal is abnormal, and
wherein the signal generation circuit is configured to generate the difference signal indicating the difference between the reset signal having the value corrected by the correction circuit and the pixel signal having the value corrected by the correction circuit.

7. The imaging system according to claim 6,
wherein the image sensor includes a target pixel and a peripheral pixel other than the target pixel,
wherein the abnormality determination circuit is configured to determine whether a value of the pixel signal output from the target pixel is abnormal using the value of the pixel signal output from the target pixel and the second reference value, and
wherein the second reference value is the value of the pixel signal output from the peripheral pixel.

8. The imaging system according to claim 6,
wherein the image sensor is configured to output the reset signal and the pixel signal in each of two or more frame periods including a target frame period,
wherein the abnormality determination circuit is configured to determine whether a value of the reset signal output in the target frame period is abnormal using the value of the pixel signal output in the target frame period and the second reference value, and
wherein the second reference value is a value of the pixel signal output in a frame period preceding the target frame period.

9. An endoscope comprising a scope configured to be inserted into a living body, the scope including:
an image sensor arranged on a distal end of the scope; and
a connector connected to the image sensor through a signal line,
wherein the image sensor includes:
two or more pixels, each of which is configured to output a reset signal and a pixel signal; and
an output circuit configured to output the reset signal and the pixel signal to the signal line,
wherein the connector includes:
a reception circuit configured to receive the reset signal and the pixel signal output to the signal line;
an abnormality determination circuit configured to determine whether a value of the reset signal is abnormal using the value of the reset signal and a reference value;
a correction circuit configured to correct the value of the reset signal when the abnormality determination circuit determines that the value of the reset signal is abnormal; and
a signal generation circuit configured to generate a difference signal indicating a difference between the reset signal having the value corrected by the correction circuit and the pixel signal, and
wherein the connector is connected to a signal processing device configured to process the difference signal and output the processed difference signal to a monitor.

10. An endoscope system comprising:
a scope configured to be inserted into a living body; and
a signal processing device,
wherein the scope includes:
an image sensor arranged on a distal end of the scope; and
a connector connected to the image sensor through a signal line,
wherein the image sensor is configured to output a reset signal and a pixel signal to the signal line,
wherein the connector includes a reception circuit configured to receive the reset signal and the pixel signal output to the signal line, and
wherein the signal processing device includes:
an abnormality determination circuit configured to determine whether a value of the reset signal is abnormal using a reference value;
a correction circuit configured to correct the value of the reset signal when the abnormality determination circuit determines that the value of the reset signal is abnormal;
a signal generation circuit configured to generate a difference signal indicating a difference between the reset signal having the value corrected by the correction circuit and the pixel signal; and
a signal processing circuit configured to process the difference signal and output the processed difference signal to a monitor.

11. An imaging method comprising:
sequentially outputting a reset signal and a pixel signal output from each of two or more pixels included in an image sensor to a signal line;
receiving the reset signal and the pixel signal output to the signal line;
determining whether a value of the reset signal is abnormal using a reference value;
correcting the value of the reset signal when it is determined that the value of the reset signal is abnormal; and
generating a difference signal indicating a difference between the reset signal having the corrected value and the pixel signal.
